(19) United States
(12) Patent Application Publication
(10) Pub. No.: US 2022/0047737 A1
(43) Pub. Date: Feb. 17, 2022

(54) BIN DISINFECTION SYSTEM

(71) Applicant: PrimeFlight Aviation Services, Inc., Sugar Land, TX (US)

(72) Inventor: Edward Zwirn, Sugar Land, TX (US)

(21) Appl. No.: 17/399,550

(22) Filed: Aug. 11, 2021

Related U.S. Application Data
(60) Provisional application No. 63/064,015, filed on Aug. 11, 2020.

(51) Int. Cl. A61L 2/10; A61L 2/26

(52) U.S. Cl. CPC A61L 2/10; A61L 2/26; A61L 2202/14; A61L 2202/20; A61L 2202/16

(57) ABSTRACT

A disinfection system for disinfecting at least one bin, comprises a housing, a lifting mechanism, at least one UV-C light and a pair of upper brackets. The housing is defined by an open top and an access opening. The lifting mechanism moves the at least one bin in a vertical plane. The UV-C light is disposed within the housing along a pathway in which the bin is moved by the lifting mechanism. A pair of catches are configured to hold the bin in suspension above the open top after being disinfected.

### CROSS-REFERENCE TO RELATED APPLICATIONS

This application claims priority to U.S. Provisional Patent Application No. 63/064,015 filed on Aug. 11, 2020, the contents of which are incorporated herein.

### FIELD

The present disclosure relates generally to a system for disinfecting reusable bins, trays and/or containers that may be used in a variety of applications.

### BACKGROUND

Infectious disease transmission is a significant personal and public health concern. Common and potentially serious viral (e.g., COVID-19, Influenza), bacterial (e.g., methicillin resistant Staph aureus), and fungal pathogens can be transmitted through the air and from mutually contacted surfaces. Many different industries use bins, trays or containers to separate and transport articles and thus can pass potential pathogens to multiple articles that occupy the bins, thereby further spreading the potential pathogens. For example, in the airline industry, carryon luggage and personal items are placed in screening bins that then pass through an X-ray scanner. While the items are subsequently removed from the bins after screening, the bins are not cleaned or disinfected between uses, despite containing personal items from passengers and the fact that passengers have handled the bins. Similarly, bins in cafeterias, or bins in distribution centers may be exposed to different potential pathogens between uses.

While different disinfecting sprays and washes may be employed between uses, such actions are time consuming and lead to delay. Ultraviolet technologies are also effective to disinfect and sanitize surfaces, but difficult and time consuming to employ for individual articles. Thus, a system that would allow contact-free movement of articles through a disinfection system, is needed.

### SUMMARY

A disinfection system for disinfecting a bin is provided. In one aspect, the disinfection system is configured to disinfect a plurality of bins stacked on top of each other. The disinfection system includes a housing defined by an open top and an access opening. The disinfection system further includes a lifting mechanism, at least one UV-C light, and a catch. The lifting mechanism is configured to move the at least one bin along a vertical plane. The UV-C light is disposed within the housing along a pathway in which the bin is moved such that the UV-C light is configured to be directed on the bin as the bin is moved along the vertical plane by the lifting mechanism. The catch is disposed adjacent the open top. The catch is configured to hold the at least one bin in suspension.

In one aspect, the lifting mechanism includes a pair of lifting elements disposed in an opposing manner to one another so as to support opposite sides of the bin. In such an aspect, the lifting mechanism further includes an engagement flange. The engagement flange is disposed on the lifting elements. The engagement flange is configured to engage a portion of the bin to move the bin in the vertical plane.

In another aspect, the catch is a pair of catches. Each catch in the pair of catches is disposed in an opposing manner to the other so as to support opposite sides of the bin. The catches may have a support surface. The support surface is movable between a retracted position and an exposed position, wherein in the exposed position, the support surface is configured to engage a portion of the bin.

In yet another aspect, the disinfecting system further includes guide elements disposed along a base member of the housing and arranged in an opposing manner. The guide elements are configured to direct the bin into the housing. In particular, the guide elements are configured to register the bin in position to be engaged by the lifting mechanism.

In yet another aspect, the disinfecting system further includes a first sensor configured to detect the presence of the at least one bin. In such an aspect, the first sensor is positioned above the open top.

In yet another aspect, the UV-C lights are angled to direct light both upwardly and downwardly. The UV-C lights may be detachably secured to sockets so as to position the UV-C lights to direct light onto the entire outer surface of the bin. The sockets may further allow for the UV-C lights to be replaced in the event the UV-C lights are burnt out or a different UV-C light or other light source is desired.

In one aspect, the access opening has a height and width configured to receive a stack of bins.

In yet another aspect, the disinfection system includes an input configured to adjust a rate of travel of the lifting mechanism.

In yet another aspect, the disinfection system further includes a cover. The cover is configured to close the open top. In such an aspect, the cover may be in the form of a roll of material. The disinfection system may further include a rolling mechanism. The rolling mechanism is configured to pull the cover over the open top, and retract the cover onto the roll of material is as to open and close the open top.

In yet another aspect, the disinfection system may further include a feeder configured to introduce the at least one bin into the access opening. In such an aspect, the feeder includes a base and a pushing mechanism. The pushing mechanism includes a plurality of links rotatably coupled together so as to move between a nested position and an extended position. In the nested position the plurality of links are pressed together and in the extended position, the plurality of links are extended.

### BRIEF DESCRIPTION OF THE DRAWINGS

At least one embodiment of the present disclosure will now be described by way of example, in greater detail with reference to the attached figures, in which:

FIG. 1 is a forward perspective view of a disinfection system for use with one or more bins;

FIG. 2 is a rear perspective view of the disinfection system of FIG. 1;

FIG. 3 is a rear perspective view of the disinfection system of FIG. 1 showing the panels removed;

FIG. 4 is a forward perspective view of the disinfection system of FIG. 1 showing lifting mechanism and the catches;

FIG. 5 is a bottom plan view of the disinfection system of FIG. 1;

FIG. 6 is a side view of a feeder for use with the disinfection system of FIG. 1;

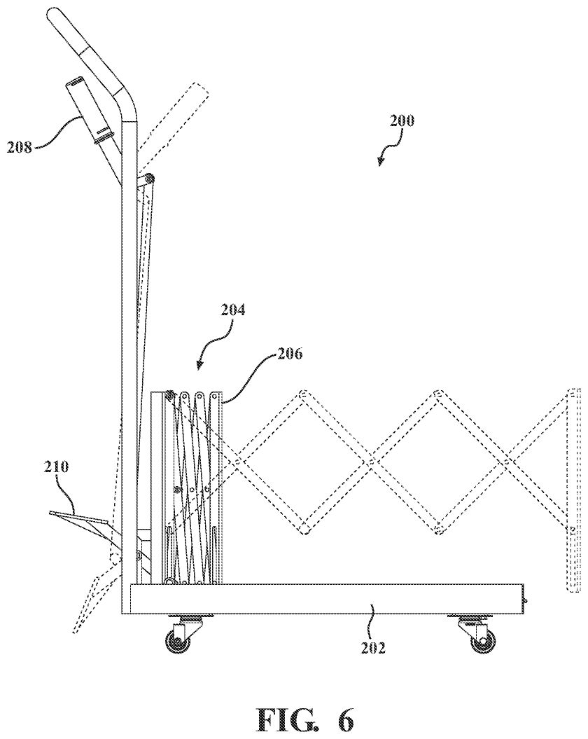

FIG. 7 is a perspective view of the feeder having a stack of bins prior to introduction into the disinfection system of FIG. 1;

FIG. 8 is a view of the feeder in FIG. 7 introducing the stack of bins into the disinfection system of FIG. 1;

FIG. 9 is a view showing the stack of bins disposed within the disinfection system of FIG. 1;

FIG. 10 is an illustrative view showing the operation of the disinfection system of FIG. 1; is a rear perspective view of the disinfection system of FIG. 4 with the bins loaded therein; and

FIG. 11 is perspective view of the cover in a retracted position;

### DETAILED DESCRIPTION

Referring now to the discussion that follows and also to the drawings, illustrative approaches to the disclosed systems and methods are shown in detail. Although the drawings represent some possible approaches, the drawings are not necessarily to scale and certain features may be exaggerated, removed, or partially sectioned to better illustrate and explain the present disclosure. Further, the descriptions set forth herein are not intended to be exhaustive or otherwise limit or restrict the claims to the precise forms and configurations shown in the drawings and disclosed in the following detailed description.

A disinfection system for disinfecting a bin is provided. The disinfection system is preferably implemented in high traffic environments such as an airport security line. The disinfection system is configured to disinfect a plurality of bins automatically and minimize manual labor so as to provide clean bins without disrupting the security process. In one aspect, the disinfection system is configured to disinfect a plurality of bins stacked on top of each other. The disinfection system includes a housing defined by an open top and an access opening. The disinfection system further includes a lifting mechanism, at least one UV-C light, and a catch. The lifting mechanism is configured to move the at least one bin along a vertical plane. The UV-C light is disposed within the housing along a pathway in which the bin is moved such that the UV-C light is configured to be directed on the bin as the bin is moved along the vertical plane by the lifting mechanism. The catch is disposed adjacent the opening. The catch is configured to hold the at least one bin in suspension.

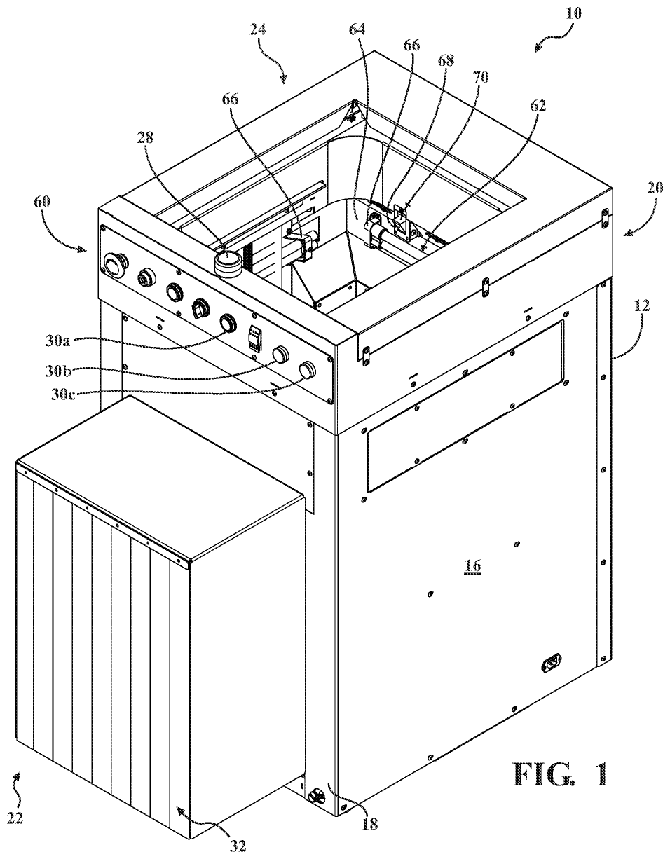

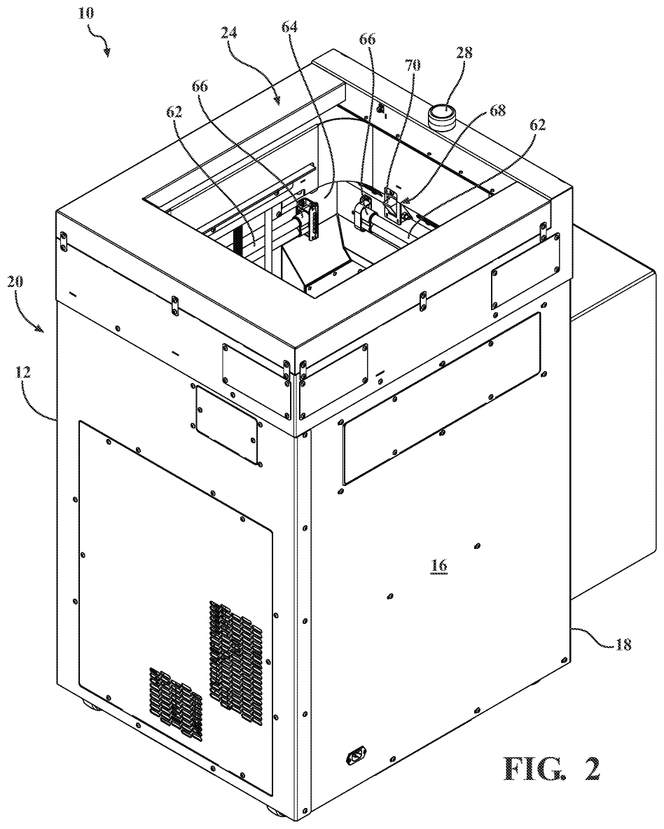

Referring to FIGS. 1-5 and 7-10, a disinfection system 10 for disinfecting bins 100, trays, or containers (hereinafter referred to as bins 100) is illustrated. The disinfection system 10 includes a front panel 12, side panels 14, 16, a rear panel 18. The front panel 12, side panels 14, 16 and rear panel 18 collectively comprise a housing 20 having an access opening 22 and a generally open top 24. An electrical compartment 26 is mounted to one of the side panels 16. The electrical compartment 26 contains the electronics needed to operate the disinfection system 10.

Above the access opening 22 in the rear panel 18, are indicator lights 28 and 30a-30c. As will be explained in further detail below, indicator lights 28 and 30a-30c signal to a user when bins 100 may be loaded into the disinfection system 10 for a disinfecting operation. In one exemplary configuration, indicator light 28 is configured to indicate that the status of the system 10, e.g. if the system is operating or not. The indicator lights 30a-30c may indicate the various aspects of the system. For instance, indicator light 30a may be actuated to indicate that the UV-C lights are operating, light 30b may indicate that the system 10 is ready to receive bins 12 and light 30c may be illuminated to indicate that the UV-C lights need to be checked, or replaced.

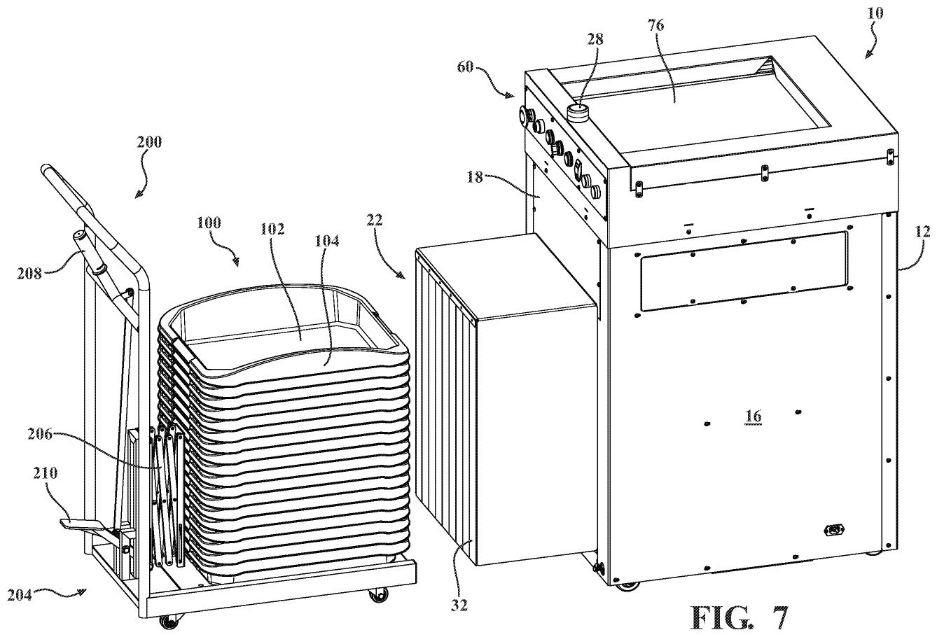

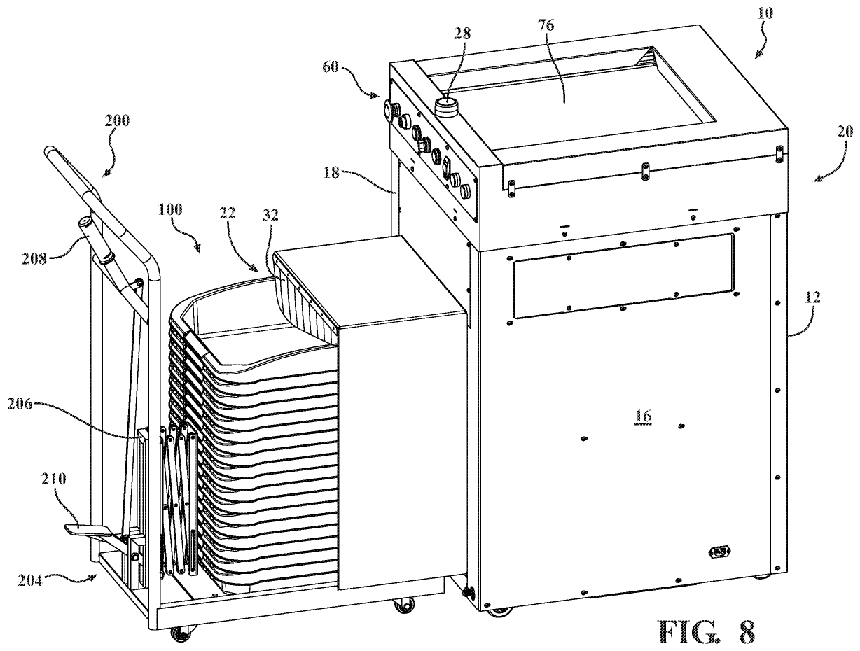

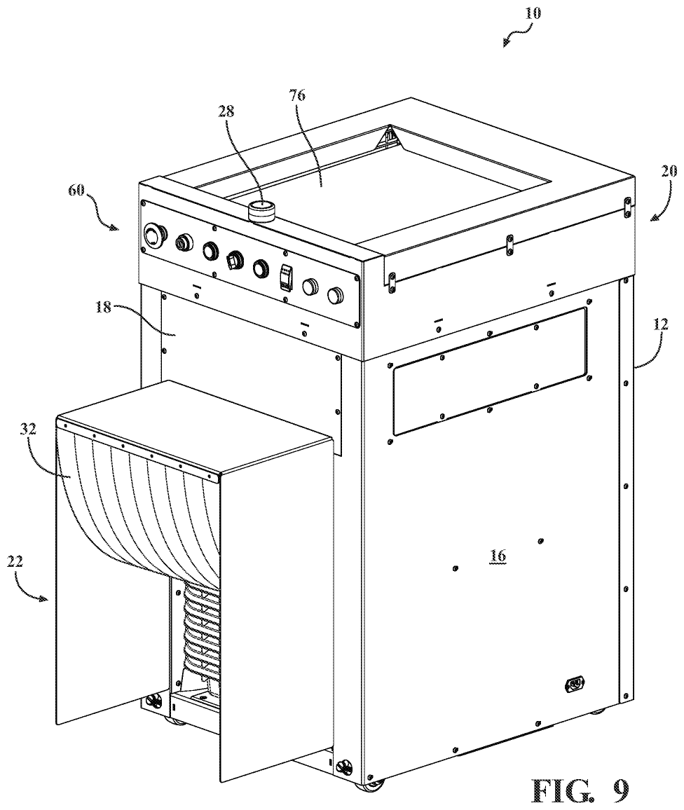

The access opening 22 is sized to accept bins 100, as shown in FIGS. 7-10. For illustrative purposes the bins 100 are shown as having a receiving body 102 and a lip 104 extending around the periphery of an opening 104 of the receiving body 102. In one exemplary arrangement, the access opening 22 is configured to accept a stack of a predetermined number of bins 100. Positioned adjacent a top portion of the access opening 22 is a UV curtain 32. UV curtain 32 serves to protect adjacent bystanders from UV lights positioned within the housing 20, as will be explained in further detail below. Preferably, the UV curtain 32 is formed of an opaque and flexible material which yields to the introduction of the bins 100 and closes the access opening 22 when the bins 100 have passed, as illustratively depicted in FIGS. 8 and 9.

To facilitate positioning the bins 100 in the housing 20, positioned adjacent a base member 34 is a guide element 36. Preferably, the housing 20 includes a pair of guide elements 36. Guide elements 36 are positioned to be generally parallel to internal surfaces of side panels 14, 16. Each guide element 36 includes an angled section 38 and a centering section 40. The angled section 38 narrows from the opening of the access opening 22 to the inner spaces of the housing 20. The centering sections 40 are preferably fixed along an axis so as to be spaced apart from each other at a fixed distance. The angled section 38 functions to center the stack of bins 100 within the housing 20 and in between the centering sections 40. In operation, the angled sections 36 direct the bins 100 centrally within in the housing 20, with the centering sections 40 serving to maintain the orientation of the bins 100.

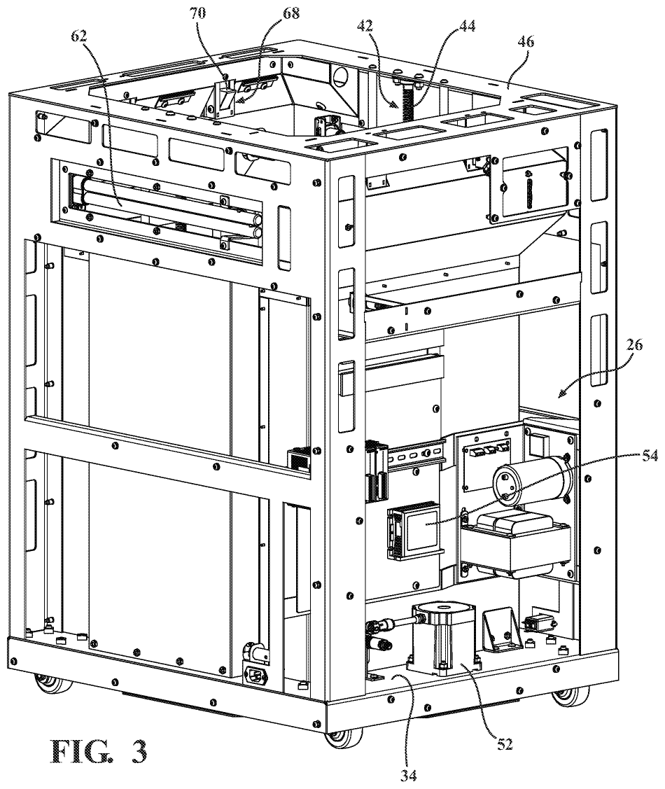

The disinfection system 10 further includes a first lifting mechanism 42. The lifting mechanism 42 is configured to engage the stack of bins 100 and move the stack of bins 100 up and down along a vertical plane. An exemplary depiction of a lifting mechanism 42 may be seen in FIGS. 3-5 and 10. In one exemplary arrangement, the first lifting mechanism 42 includes a pair of guide rails 44 positioned between the guide element 36 and an interior surface of the side panels 14, 16. The guide rails 44 extend vertically from the base member 34 to a bottom surface of a land member 46 that frames the open top 24.

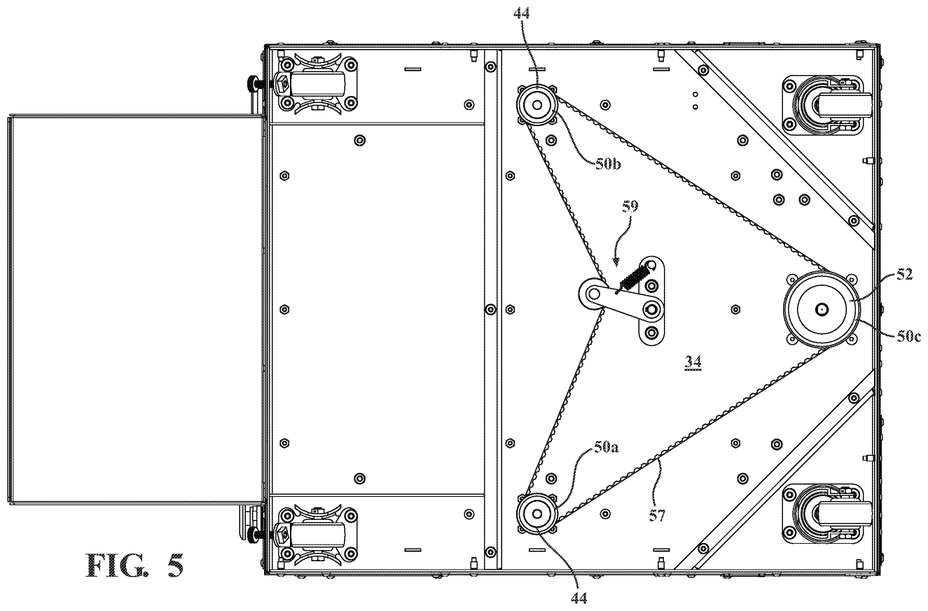

The guide rails 44 are threaded members which may be rotated by a pulley system 48 arranged at the bottom of the housing 20. As shown in FIG. 5, a pair of gears 50a, 50b are fixed to a respective guide rail 44 and may be turned clockwise or counterclockwise by a motor 52. The motor 52 is coupled to a driving gear 50c. The motor 52 may be controlled by a Programmable Logic Controller (“PLC”) 54 (shown in FIG. 3). The motor 52 and the driving gear 50c rotates a belt 57 which is coupled to the respective gears 50a, 50b so as to turn the guide rails 44 in a clockwise or counterclockwise direction. A tensioning element 59 is coupled to the belt 57 so as to maintain a tension on the belt 57 and assist in the rotation of the gears 50a, 50b.

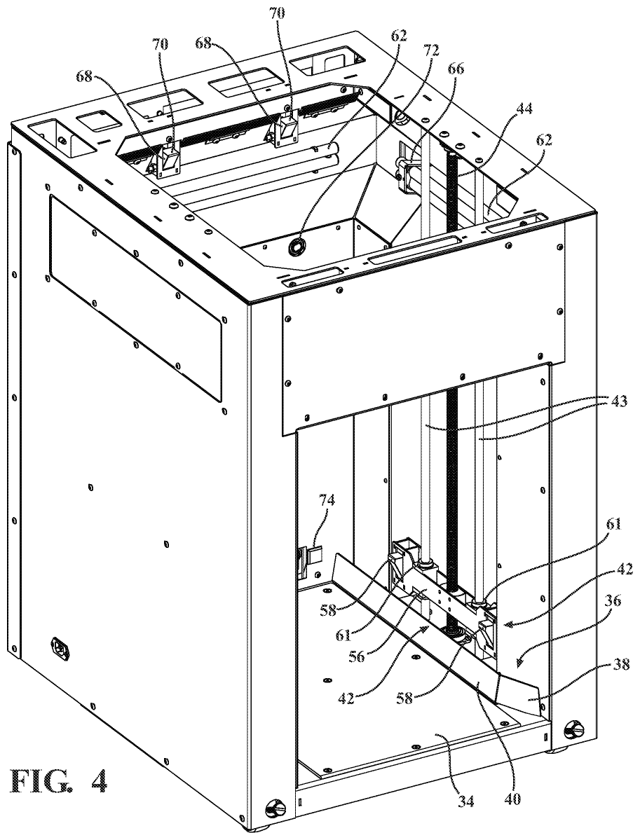

As shown in FIG. 4, a lifting element 56 is disposed on each of the guide rails 44 with the lifting element 56 being selectively moveable in the vertical plane, as will be described below in further detail below. The lifting element 56 is a generally elongated member and may include a pair of through-holes 61 forming a sleeve slidably coupled to a pair of support rods 43. The support rods 43 are on opposite sides of a corresponding guide rail 44 so as to prevent the lifting element 56 from being cantilevered. Each lifting element 56 may further includes an engagement flange 58 that extends laterally away from respective side panels 14, 16, toward an interior of the housing 20. Each lifting element 56 further a threaded bore 58 threadedly engaged to a respective guide rail 44.

The lifting mechanism 42 depicted herein is provided for illustrative purposes and is not limiting to the scope of the appended claims. Any lifting mechanism currently known or later developed, which performs the same operation as described herein, e.g. lifting the bins through a UV-C light source and exposing the bottom surface of the bins to the UV-C light may be adapted for use herein. The lifting mechanism 42 is illustratively shown as being powered by an external power source, such as a standard commercial or residential outlet. However, it should be appreciated that the lifting mechanism 42 may be configured to be powered by an external or internal rechargeable battery (not shown). The PLC 54 may be programmed for an automated operation, as discussed in more detail below. The housing 20 may include an instrument panel 60 for providing command instructions to the lifting mechanism 42, such as stop, or increase or decrease the speed at which the guide rails 44 are turned, increasing or decreasing the speed at which the bins are moved up or down, as the case may be. These commands may be inputted via a button or a dial. The indicating lights 28, 30a-30c may be disposed on the instrument panel 60.

In operation, the driving gear 50c is attached to the motor 52 that is operatively connected to a power source. As the motor 52 rotates, the driving gear 50c rotates and thus rotates the belt along the other gears 50b, 50c which turn the guide rails 44. Both guide rails 44 rotate in response to rotation of the gears 50b, 50c. Rotation in a first direction will cause the lifting element 56 to move upwardly along the guide rails 44, while rotation in a second direction will cause the lifting element 56 to move downwardly along the guide rails 44.

Disposed within the housing 20 are UV-C lights 62. When activated, UV-C lights 62 serve to disinfect bins 100. In one exemplary configuration, there is a pair of UV-C lights 62 mounted on each side panel 14, 16, the rear panel 18, and front panel 12. To insure that disinfecting light from UV-C lights 62 shine on all surface of bins 100, in one exemplary arrangement, UV-C lights 62 are arranged to be slightly angled with respect to each other, such that light is directed downwardly by the upper light and light is directed upwardly by the lower light.

As an alternative, or in addition to angling the UV-C lights 62, the side panels 14, 16, front and rear panels 12, 18 may include an inset section 64 with reflective surfaces that include angled sections to direct light in different directions. The UV-C lights 62 may be detachably secured to sockets 66 so as to position the UV-C lights 62 to direct light onto the entire outer surface of the bin 100. The sockets 66 may further allow for the UV-C lights 62 to be replaced in the event the UV-C lights 62 are burnt out or a different UV-C light 62 or other light source is desired.

Positioned adjacent land member 46 on an interior surface of side panels 14, 16 are catches 68. In one exemplary arrangement, each side panel 14, 16 includes a pair of catches 68 positioned on either side of the guide rails 44. Each catch 68 includes a support surface 70 on its top surface that is configured to engage a portion of a bin 100, as will be explained below. In one exemplary arrangement, the catches 68 are configured as spring loaded members that are biased in an exposed position (as shown in FIG. 4). In the exposed position, the support surface 70 is exposed and extends toward the open area in the open top 24.

In operation, the support surface 70 is moved to the retracted position by an engagement of the lip 104 of the bin 100. In particular, as the lifting mechanism 42 lifts the bin 100 upwardly from the base 34 of the housing 20, the lip 104 pushes the catches inwardly. When the lip 104 passes the catches 68, the catch 68 is free to move to the exposed position. In such a manner, the bins 100 may be positioned above the support surface 70 and is held in suspension by the support surface 70 with gravity assist.

Referring to FIGS. 7-11, an operation of the disinfection system 10 will now be described. First, at least one bin 100, and in one exemplary arrangement, a stack of bins 100 is inserted through access opening 22 in the rear panel 18. In one aspect, a feeder 200 may be used to insert the stack of bins 100. The feeder 200 includes a base 202 which may be attached to wheels to allow for portability of the bins 100. The feeder 200 includes a pushing mechanism 204. The pushing mechanism 204 may include a plurality of links 206 rotatably coupled to each other. A release 208 and a foot pedal 210 may be mechanically attached to the plurality of links 206. The release 208 is configured to release the foot pedal 210 wherein an actuation of the foot pedal 210 actuates the links 206 so as to extend outwardly (shown in phantom) and feed the stack of bins 100 into the housing 20. More specifically, bins 100 are pushed along base member 34. The UV curtain 32 is preferably flexible such that when the bins 100 are fully seated within the housing 20, the curtain overhangs the access opening 22. The number of bins 100 that can be seated within the disinfection system 10 is predetermined, and in one exemplary arrangement, based upon the height of access opening 22.

In one exemplary arrangement, the indicator lights 28 and 30a and 30b are activated when the disinfection system 10 is turned on. More specifically, indicator light 30b is turned on indicating that the bins 100 may be inserted into disinfection system, upon the bins 100 being properly inserted into the housing 20, the lifting mechanism 42 and the UV-C lights 62 may be turned on and thus indicator light 28 and 30c are actuated. This may be helpful to the user as the UV-C light 62 is predominantly obstructed by the UV curtain 32.

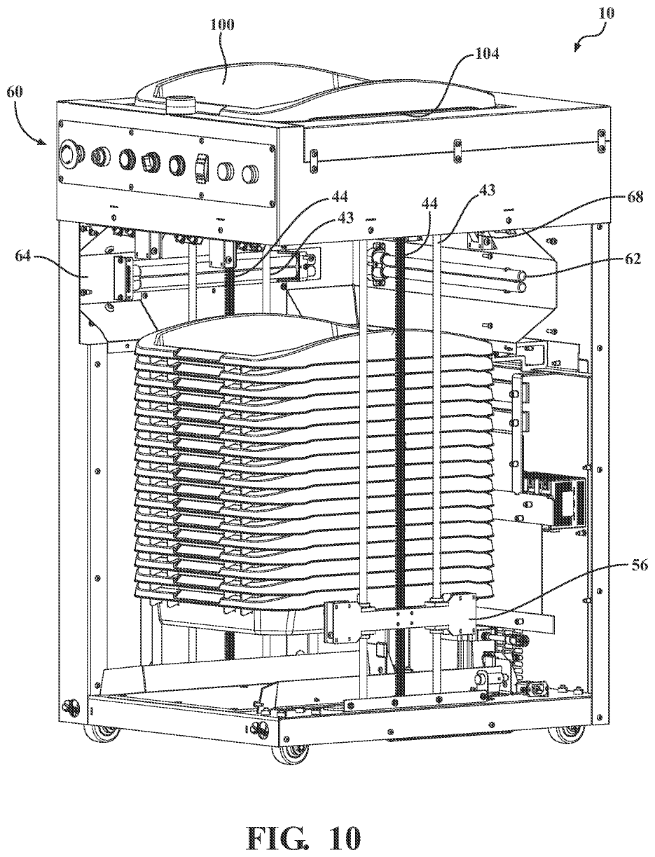

The bins 100 nest together to form a stack. Referring to FIGS. 9 and 10, once the stack of bins 100 is fully seated within the disinfection system 10, the UV-C lights 62 are turned on. This may be done automatically upon detection of the bins 100 within the housing 20. Due to the positioning of the UV-C lights 62, at least a portion of light emitting from the UV-C lights 62 will be directed downward toward the inner surface of the receiving body 102 and the top surface of the lip 104 of the uppermost bin 100 in the stack.

Next, the first lifting mechanism 42 may be activated. More specifically, in an initial position, the engagement flanges 58 of the lifting elements 42 are disposed beneath the lowermost lip 104 of the stack of bins 100. Once the lifting mechanism 42 is activated, the engagement flange 58 lifts the stack of bins 100 at a predetermined speed upwardly until the uppermost bin 100 overcomes the biasing force of the catches 68 such that the first bin 100 is moved above the catches 68 and hooked into open top 24. Once in this position, the remaining bins 100 in the stack are lowered back down below the UV-C lights 62 by moving the lifting elements 56 downwardly.

The PLC 54 may be programmed to execute the raising and lowering of the stack of bins 100 using sensors to detect the position of the bins. For instance, a first sensor 72 may be provided adjacent the land member 46 to detect a bin 100 within the open top 24. Alternatively, the first sensor 72 may be configured to detect the change in the catch 68 from an exposed position, to a retracted position and then to the exposed position, indicating that a lip 104 of the top most bin 100 has engaged the catch 68. It should be appreciated that the determination of a top most bin 100 engaged or otherwise suspended by the catches 68 may be done in numerous ways and is not limiting, and said determination may be used by the PLC 54 to actuate the lift mechanism 42 to lower the stack of bins 100. When the top most bin 100 is suspended by the catches 68, the bottom surface of the receiving body 102 and the lip 104 of the suspended bin 100 are exposed to UV-C lights 62 and disinfected. Concurrently, the top surface of the receiving body 102 and the top surface of the receiving body 102 and the lip 104 of the top most bin 100 in the stack are disinfected by the UV-C lights 62.

The disinfection system 10 repeats the above process, one bin 100 at a time at a certain speed to achieve effective disinfection. When completed, at least a portion of the stack of bins 100 will be disposed above the open top 24 and ready for a user to remove an individual bin 100 for use. This system 10 is particularly useful at a security check point, where users are asked to place their belongings into a bin 100 for screening. When the stack of bins 100 have all been moved to the catches 68, the lifting mechanism 42 returns to the initial positon to be fed with another stack of bins 100 and the indicator light 28 is turned on.

In one exemplary arrangement, the first sensor 72 and a second sensor 74 are provided to facilitate operation of disinfection system 100. In one exemplary configuration, the first sensor 72 and the second sensor 74 may be light sensors, although other sensors may be employed without departing from the disclosure. The First sensor 72 may be positioned adjacent a top portion of the housing 20. The first sensor 72 is configured to detect the presence of a top bin 100. The number of detected bins 100 may be counted by the PLC 54, wherein the PLC 54 may be operable to stop the lifting mechanism 42 after a predetermined number of bins 100 have been placed in suspension by the catches 68. Such an operation may be helpful in pausing the disinfection system 10 such that no more bins 100 are transported upwardly above catches 68. It should be appreciated that the signal from the first sensor 72 may be processed by the PLC 54 to perform other tasks to improve the efficiency of the system 10. For instance, the PLC 54 may be programmed to actuate the indictor light 30c after a set number of bins 100 have been disinfected so as to alert the user in a maintenance program. For instance, the user may be alerted to perform a service maintenance after ten thousand bins 100 have been disinfected.

The second sensor 74 may be provided to detect when no further bins 100 are present within disinfection system 10. More specifically, second sensor 74 is positioned adjacent the base member 34 and serves to turn off the UV-C lights 66 when the last bin 100 has been removed. That is, when the second sensor 74 does not detect the presence of a bin 100, the PLC 54 may be programmed to automatically stop the operation of the system 100, or to actuate light 30b, indicating that the system 10 may receive more bins 100.

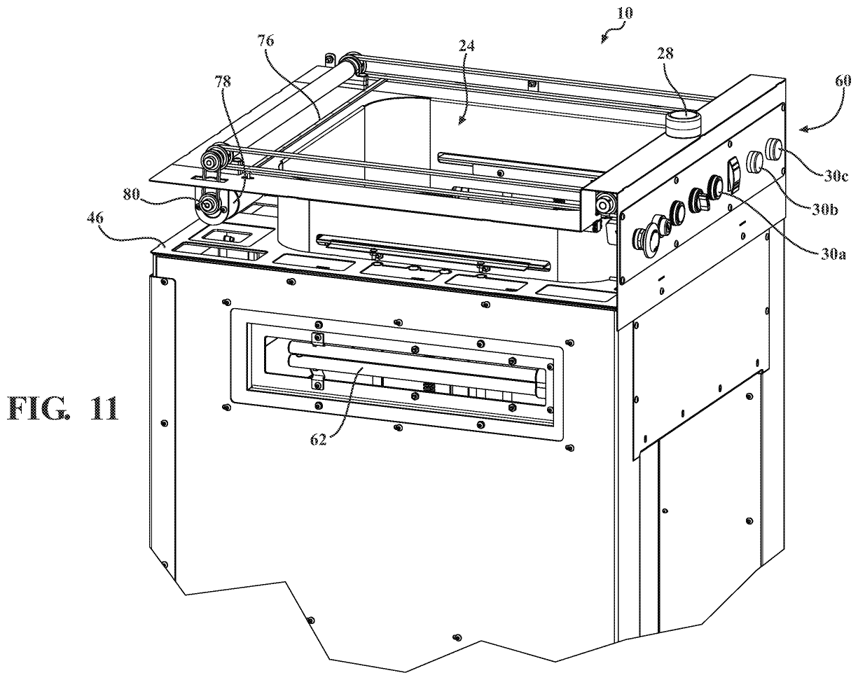

With reference now to FIG. 11, the PLC 54 may be further programmed to actuate a cover 76. The cover 76 may be configured to close the open top 24. The cover 76 is housed within the land member 46 and may be provided in a roll of material 78. The roll of material 78 is spooled about a shaft 80. The cover 76 may be pulled from the roll of material 80 across the open top 24 so as to close the open top 24 by a rolling mechanism 82. The rolling mechanism 82 may be further configured to wind or rotate the roll of material 80 so as to retract the cover 76 onto the roll of material 80 to open the open top 24.

As shown in FIGS. 7-9, the cover 76 is in a closed position, wherein light from the actuation of the UV-C lights 60 does not escape past the open top 24. An illustrative example of a rolling mechanism 82 is shown in FIG. 11, wherein a first motor 84 rotates a wheel 86 which rotates a cover belt 88 to which a lead end of the cover is fixedly attached. A second motor 88 rotates the roll of material so as to pull the cover 76. In such a manner, the cover 76 may be pulled to cover the open top 24 or the roll of material 80 may be wound so as to open the open top 24. In one aspect, the PLC 54 is programmed to actuate the rolling mechanism 82 so as to close the cover 76 when the second sensor 76 detects that there are no bins 100 in the housing 20. FIGS. 7-9 show the cover 76 in the closed position. As the bins 100 are introduced into the housing 20, the first sensor 72 detects no bins 100, and the second sensor 74 detects bins 100. The PLC 54 may processes such a detection state to actuate the rolling mechanism 82 to move the cover 76 into the open position and initiate the lifting mechanism 42 to lift the stack of bins 100 upwardly and actuate the UV-C light 62. In such a manner, the system 100 may be programmed to operate automatically, and may be programmed to automatically shut down when the first sensor 72 and the second sensor 74 detect the absence of bins 100 within the housing 20. In one aspect of a shutdown operation, the lifting mechanism is actuated so as to place lifting element 56 near the base member 34 so as to position the lifting element 56 to receive a stack of bins 100, the UV-C lights 62 are turned off and the cover 76 is moved to the closed position.

Accordingly, an illustrative depiction of a disinfecting system 100 is provided which automatically disinfects a plurality of bins in a stack of bins using UV-C light 60. Such a system 100 facilitates the security process in various applications to include an airport security checkpoint, but may be applicable to other public gathering events such as concerts, plays and the like. Other aspects of the system 10 may optionally include rollers 76 (wheels) disposed on the bottom surface of the housing 20 to allow the system 10 to be portable.

The following Clauses provide exemplary configurations for a disinfecting system as described above.

Clause 1. A disinfection system for disinfecting system for disinfecting a bin includes a housing defined by an open top and an access opening. The disinfection system further includes a lifting mechanism, at least one UV-C light, and a catch. The lifting mechanism is configured to move the at least one bin along a vertical plane. The UV-C light is disposed within the housing along a pathway in which the bin is moved such that the UV-C light is configured to be directed on the bin as the bin is moved along the vertical plane by the lifting mechanism. The catch is disposed adjacent the opening. The catch is configured to hold the at least one bin in suspension

Clause 2. The disinfection system of Clause 1, wherein the lifting mechanism includes a pair of lifting elements disposed in an opposing manner to one another.

Clause 3. The disinfection system of Clause 2, wherein lifting mechanism further includes an engagement flange disposed on the lifting elements, the engagement flange configured to engage a portion of the bin to move the bin in the vertical plane.

Clause 4. The disinfection system of Clause 1, wherein the catch is a pair of catches, each catch in the pair of catches disposed in an opposing manner to the other, the catches having a support surface movable between a retracted position and an exposed position, wherein in the exposes position, the support surface is configured to engage a portion of the bin.

Clause 5. The disinfection system of Clause 1, further including guide elements disposed along a base member of the housing and arranged in an opposing manner, the guide elements configured to direct the at least one bin into the housing.

Clause 6. The disinfection system of Clause 1, further including a first sensor configured to detect the presence of the at least one bin.

Clause 7. The disinfection system of Clause 6, wherein the first sensor is positioned above the open top.

Clause 8. The disinfection system of Clause 1, wherein the UV-C lights are angled to direct light both upwardly and downwardly.

Clause 9. The disinfection system of Clause 1, wherein the access opening is configured to receive a stack of bins.

Clause 10. The disinfection system of Clause 1, including an input configured to adjust a rate of travel of the lifting mechanism.

Clause 11. The disinfection system of Clause 1, further including a cover configured to close the open top.

Clause 12. The disinfection system of Clause 11, wherein the cover is in a roll of material.

Clause 13. The disinfection system of Clause 12, further including a rolling mechanism, the rolling mechanism configured to pull the cover over the open top, and retract the cover onto the roll of material is as to open and close the open top.

Clause 14. The disinfection system of Clause 1, further comprising a feeder configured to introduce the at least one bin into the access opening.

Clause 15. The disinfection system of Clause 14, wherein the feeder includes a base and a pushing mechanism, the pushing mechanism including a plurality of links rotatably coupled together so as to move between a nested position and an extended position, wherein in the nested position the plurality of links are pressed together and in the extended position, the plurality of links are extended.

It is intended that the scope of the present system be defined by the following claims. However, it must be understood that this invention may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims. The scope of the invention should be determined, not with reference to the above description, but should instead be determined with reference to the appended claims, along with the full scope of equivalents to which such claims are entitled. It is anticipated and intended that future developments will occur in the arts discussed herein, and that the disclosed systems and methods will be incorporated into such future examples. Furthermore, all terms used in the claims are intended to be given their broadest reasonable constructions and their ordinary meanings as understood by those skilled in the art unless an explicit indication to the contrary is made herein. In particular, use of the singular articles such as “a,” “the,” “said,” etc. should be read to recite one or more of the indicated elements unless a claim recites an explicit limitation to the contrary. It is intended that the following claims define the scope of the invention and that the method and apparatus within the scope of these claims and their equivalents be covered thereby. In sum, it should be understood that the invention is capable of modification and variation and is limited only by the following claims.

What is claimed is:

1. A disinfection system for disinfecting at least one bin, comprising:
a housing defined by an open top and an access opening;
a lifting mechanism for moving at least one bin in a vertical plane;
at least one UV-C light disposed within the housing along a pathway in which the bin is moved such that the UV-C light is configured to be directed on the bin as the bin is moved in the vertical plane by the lifting mechanism; and
a catch disposed adjacent the opening, the catch configured to hold the at least one bin in suspension.

2. The disinfection system of claim 1, wherein the lifting mechanism includes a pair of lifting elements disposed in an opposing manner to one another.

3. The disinfection system of claim 2, wherein lifting mechanism further includes an engagement flange disposed on the lifting elements, the engagement flange configured to engage a portion of the bin to move the bin in the vertical plane

4. The disinfection system of claim 1, wherein the catch is a pair of catches, each catch in the pair of catches disposed in an opposing manner to the other, the catches having a support surface movable between a retracted position and an exposed position, wherein in the exposes position, the support surface is configured to engage a portion of the bin.

5. The disinfection system of claim 1, further including guide elements disposed along a base member of the housing and arranged in an opposing manner, the guide elements configured to direct the at least one bin into the housing.

6. The disinfection system of claim 1, further including a first sensor configured to detect the presence of the at least one bin.

7. The disinfection system of claim 6, wherein the first sensor is positioned above the open top.

8. The disinfection system of claim 1, wherein the UV-C lights are angled to direct light both upwardly and downwardly.

9. The disinfection system of claim 1, wherein the access opening is configured to receive a stack of bins.

10. The disinfection system of claim 1, including an input configured to adjust a rate of travel of the lifting mechanism.

11. The disinfection system of claim 1, further including a cover configured to close the open top.

12. The disinfection system of claim 11, wherein the cover is in a roll of material.

13. The disinfection system of claim 12, further including a rolling mechanism, the rolling mechanism configured to pull the cover over the open top, and retract the cover onto the roll of material is as to open and close the open top.

14. The disinfection system of claim 1, further comprising a feeder configured to introduce the at least one bin into the access opening.

15. The disinfection system of claim 14, wherein the feeder includes a base and a pushing mechanism, the pushing mechanism including a plurality of links rotatably coupled together so as to move between a nested position and an extended position, wherein in the nested position the plurality of links are pressed together and in the extended position, the plurality of links are extended.
